(19) United States
(12) Patent Application Publication
(10) Pub. No.: US 2015/0018694 A1
(43) Pub. Date: Jan. 15, 2015

(54) RESUSCITATION ASSEMBLY WITH PEEP VALVE

(71) Applicant: Laerdal Global Health AS, Stavanger (NO)

(72) Inventor: Øystein Gomo, Hundvag (NO)

(73) Assignee: Laerdal Global Health AS, Stavanger (NO)

(21) Appl. No.: 14/380,319

(22) PCT Filed: Feb. 27, 2013
(86) PCT No.: PCT/EP2013/053907

§ 371 Date: Aug. 21, 2014

(30) Foreign Application Priority Data
Feb. 27, 2012 (NO) 20120206

(51) Int. Cl. A61M 16/20; A61M 16/06; A61B 5/00; A61B 5/0205; A61B 5/113; A61M 16/00; A61M 16/08

(52) U.S. Cl. CPC A61M 16/208; A61M 16/0084; A61M 16/06; A61M 16/0003; A61M 16/0858; A61B 5/0205; A61B 5/113; A61B 5/742; A61M 2016/0027; A61B 5/024
USPC 600/484; 128/205.13; 600/529

(57) ABSTRACT

Resuscitation assembly (101) comprising a patient mask (107), a ventilation bag (105), an inflation valve (113), an exhalation valve (111), and an expiration indicator (119). The expiration indicator (119) is a positive end expiratory pressure valve in the form of a slit valve that exhibits a slit (131) in a flexible sheet part (125). Also disclosed is a resuscitation assembly (101) comprising a patient mask (107), a ventilation bag (105), an inflation valve (113), an exhalation valve (111), a positive end expiratory pressure valve (119), and a pressure sensor (133) adapted to measure pressure on the patient side (127) of the positive end expiratory pressure valve (119).

The present invention relates to a resuscitation assembly for artificial ventilation of the lungs of patients. In particular it regards to such an assembly which exhibits a positive end expiratory pressure valve.

### BACKGROUND

When newborn babies do not begin to breathe spontaneously right after birth, they need artificial ventilation of the lungs to survive. This also applies to older children and adults in a non-breathing, unconscious state.

Today, manual ventilations are usually given with a resuscitator. This is a device where a soft volume (usually referred to as a “silicone bag”) is squeezed by hand forcing fresh air into the mouth and lungs of the patient. Valve mechanisms guide inspiration air into the patient, while expiration air is lead out through a different exit-gate to make sure that no used air enters the silicone bag. New, fresh air is instead sucked into the silicone bag through another valve, making the resuscitator ready for the next inspiration of air.

Common types of resuscitators let the expiration air directly out to the surroundings. Thus after expiration the pressure in the lungs of the patient is approximately the same as the ambient pressure.

Newborns, particularly preterm newborns with yet not completely developed lungs should get PEEP-ventilations (Positive End Expiratory Pressure) to prevent the lungs from collapsing between each breath. This pressure should typically be in the range 5-8 cm H₂O. Today such a positive end expiratory pressure can be achieved by using an extra device arranged on the resuscitator assembly. The purpose of this is to collect the dispersed expiration air and guide it through a PEEP valve. The PEEP valve exhibits a spring-retained closing mechanism. The exhaled air must exceed the pressure required to open the valve. The required opening-pressure is determined by the force from the steel spring. A typical PEEP-valve which can be added on to a resuscitator can consist of 6-8 parts. This makes it expensive and cumbersome to clean.

One of the greatest challenges with lung ventilations today is to get a good seal between the patient mask and the face of the patient. Leakage here reduces the amount of air or prevents air from entering the lungs. Air leakages can also occur in the so called pop-off valve which is standard on resuscitators for newborns. The pop-off valve is a pressure relief valve that prevents excessive pressure to be induced in the lungs.

Another reason why air is prevented from entering the lungs is if the head is not positioned correctly to give an open airway. Ineffective ventilations can be hard to spot and can be critical for the patient.

There is therefore a need for an indication of effective ventilation.

### THE INVENTION

According to a first aspect of the present invention there is provided a resuscitation assembly comprising a patient mask, a ventilation bag, an inflation valve, an exhalation valve, and an expiration indicator. The expiration indicator is a positive end expiratory pressure valve (PEEP valve). Furthermore, the expiration indicator is a slit valve that exhibits a slit in a flexible sheet part.

As used herein, a PEEP valve shall be understood as a valve through which the exhaled air of the patient is guided, and which retains a pressure in the lungs of the patient after exhalation. Thus, the PEEP valve will close when the pressure on a patient side of the valve drops to below a threshold value. In order to let exhalation air out of the resuscitation assembly, it will of course open at a given pressure.

The sheet part is sufficiently thin to be flexed by a pressure resulting from exhalation of the patient, and simultaneously sufficiently thick to provide some resistance before being flexed.

The expiration indicator has a patient side and an ambience side. The patient side is the side of the expiration indicator from which exhaled air flows through it.

The opposite side is the ambience side, to which side the exhaled air is guided when flowing through the expiration indicator.

The flexible sheet part can preferably exhibit a convex face that faces the patient side. In this embodiment the flexible sheet part is adapted to deflect towards the ambience side at a given pressure on the patient side in such way that a visible gap is formed in the slit. The term convex shall be interpreted in a broad sense. For instance, a pyramid shape or a shape with two plane faces having an angle between them, or a dome shape shall be covered by the term.

The resuscitation assembly according to an embodiment of the present invention comprises a valve assembly arranged between the patient mask and the ventilation bag. In this embodiment, the expiration indicator can advantageously be arranged to the valve assembly.

Furthermore, the valve assembly can exhibit a pipe stub which is adapted to receive the expiration indicator.

The expiration indicator can be in the form of a cap which is adapted to be releasably arranged to a receiving connecting piece of the resuscitation assembly, such as the pipe stub mentioned above.

Such a cap can exhibit a circular body with a circular protrusion or a circular groove arranged in the inwardly facing face of said circular body. With such an embodiment the cap (i.e. the expiration indicator) can easily be attached to a facing groove or protrusion, respectively, of the resuscitation assembly, for instance on the pipe stub.

Thus, an expiration indicator is provided which is cheap to manufacture and which easily can be replaced. It shall also be noted that the resuscitation assembly will function also when the expiration indicator is not present, of course then however without the functions of the expiration indicator/PEEP valve.

In an advantageous embodiment of the first aspect of the invention, the resuscitation assembly comprises a pressure sensor which is adapted to measure pressure on a patient side of the expiration indicator. In one embodiment the measured pressure values, as measured by said pressure sensor, can be to be communicated wirelessly to a remote receiver. For performing this function the resuscitation assembly can comprise a signal transmission device and a battery.

Moreover, the remote receiver can be adapted to receive and to display a plurality of different measured patient parameters, such as said pressure values, heart rate values, and chest rise values.

Thus, according such an embodiment of the first aspect of the invention, the person performing the ventilation with the resuscitation assembly is able to detect a malfunctioning seal between the mask and the face of the patient both by means of the expiration indicator and by means of the measured pressure values which is displayed to him or her.

According to a second aspect of the present invention there is provided a resuscitation assembly comprising a patient mask, a ventilation bag, an inflation valve, an exhalation valve, and a positive end expiratory pressure valve. According to the second aspect of the invention the assembly further comprises a pressure sensor which is adapted to measure pressure on the patient side of the positive end expiratory pressure valve.

In one embodiment of the second aspect of the invention the resuscitation assembly comprises a signal transmission device which is adapted to transmit measured pressure values to a remote receiver. Advantageously it further comprises a display adapted to display pressure values, as measured by said pressure sensor.

The resuscitation assembly according to the various aspects of the invention thus combines the function of retaining a minimum air pressure in the lungs of the patient and the function of giving a visual feedback on effective ventilations.

It should be understood that while the present invention is particularly useful for use with newborns, it may also be useful with other types of patients, as indicated above.

### EXAMPLE OF EMBODIMENT

While the invention has be described in general terms above, a more detailed example of embodiment will now be described with reference to the drawings, in which

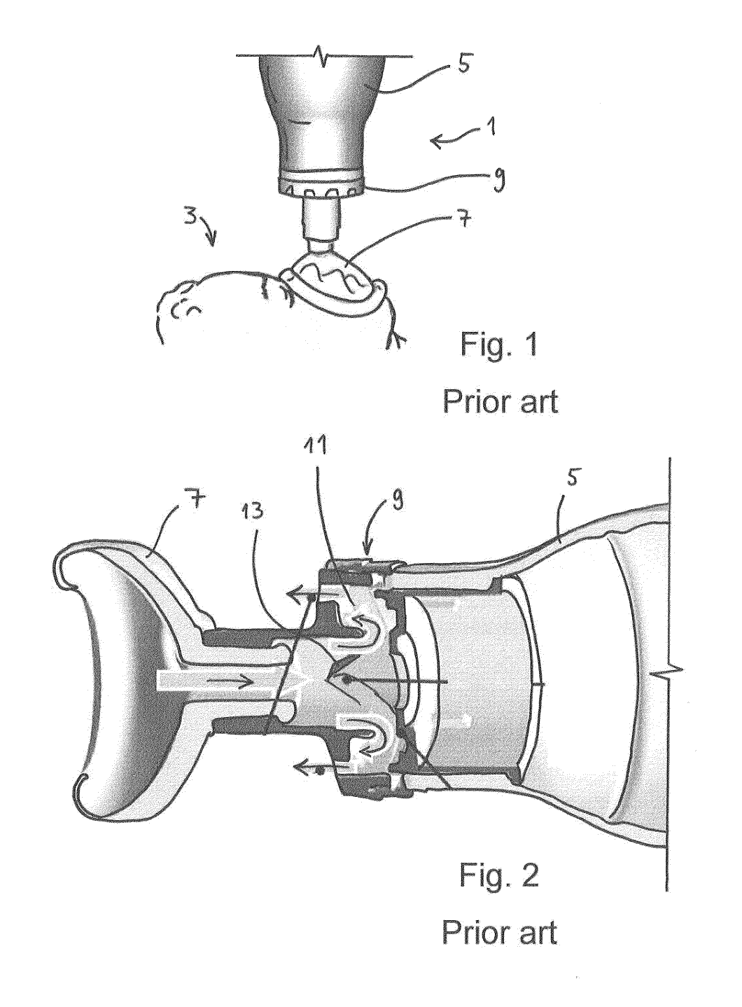

FIG. 1 is a side view of a ventilation assembly of the prior art in use on a patient;

FIG. 2 is a cross section view showing the valve assembly of a common ventilation assembly of the prior art;

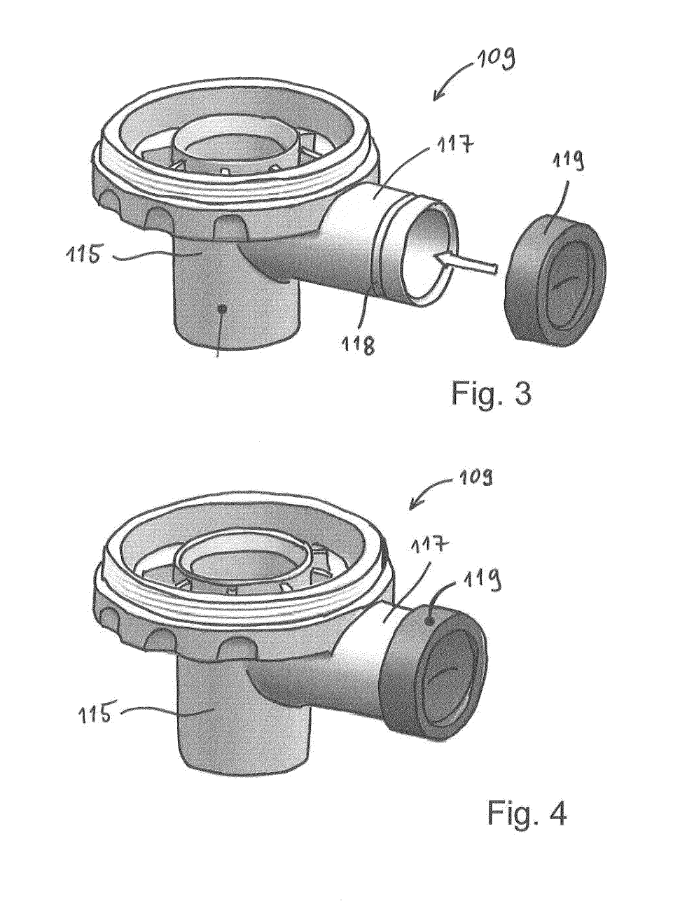

FIG. 3 is a perspective view of a valve assembly and an expiration indicator, being part of a resuscitation assembly according to the present invention;

FIG. 4 is a perspective view corresponding to FIG. 3, however with the expiration indicator attached to the valve assembly;

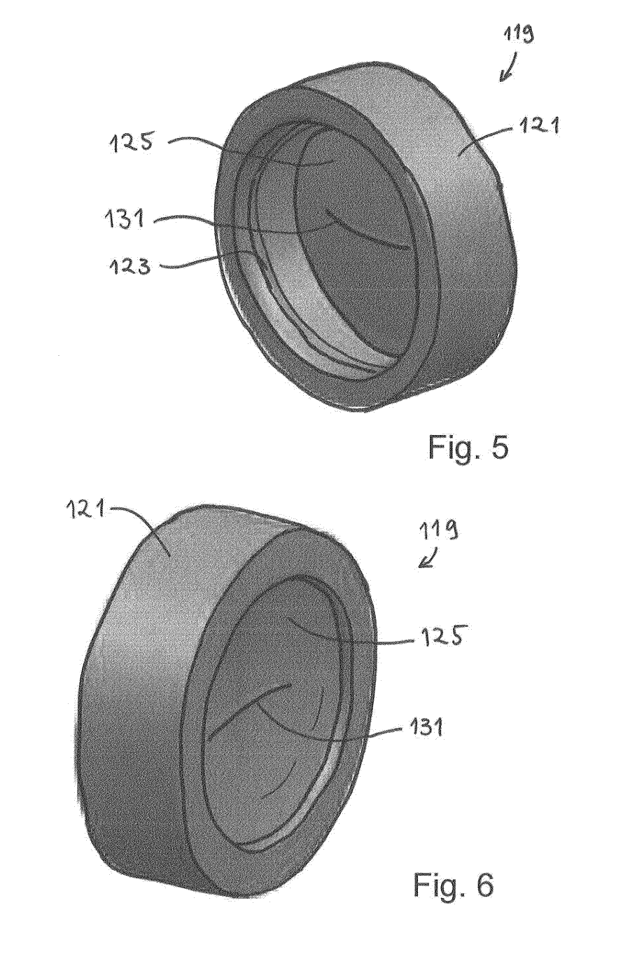

FIG. 5 is an enlarged perspective view of the expiration indicator shown in FIG. 3 and FIG. 4;

FIG. 6 is another enlarged perspective view of the expiration indicator;

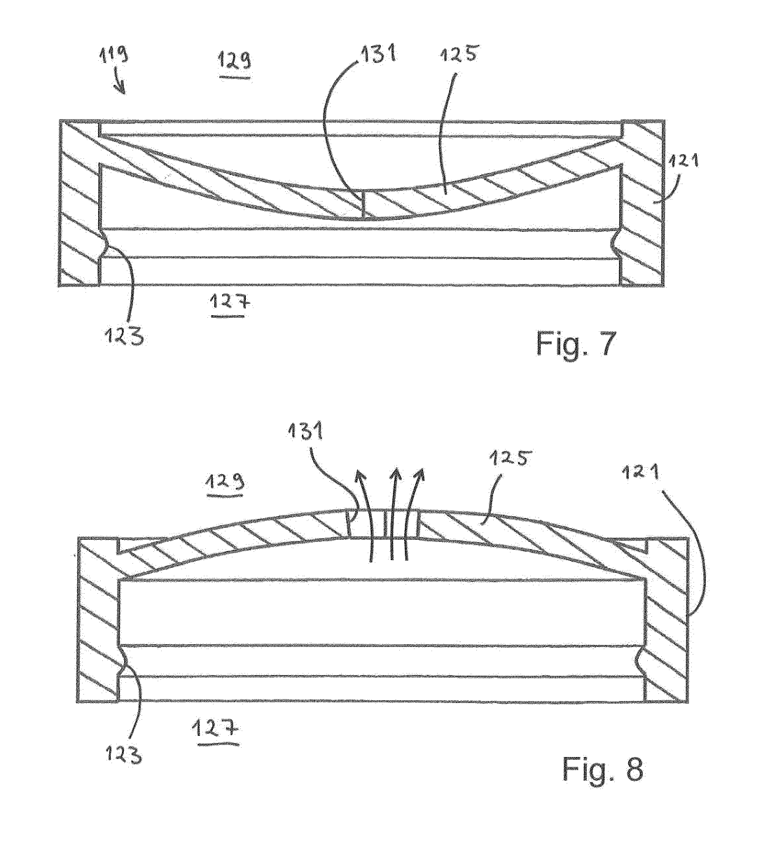

FIG. 7 is a cross section view of the expiration indicator in a closed mode;

FIG. 8 is a cross section view of the expiration indicator in an open mode;

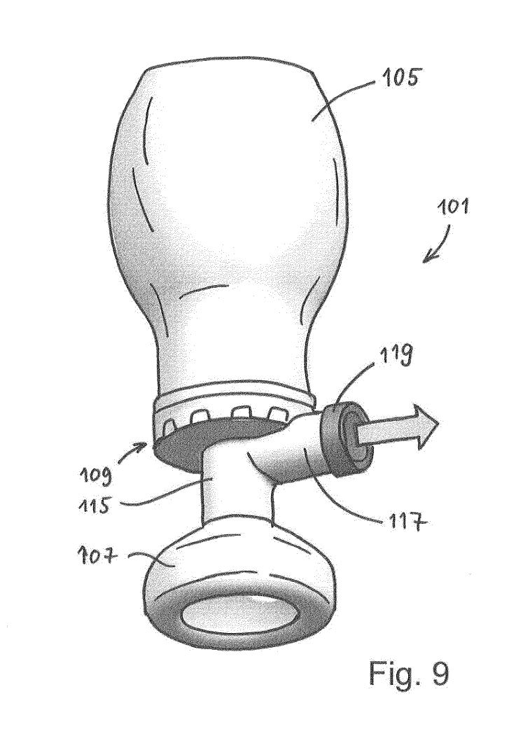

FIG. 9 is a perspective view of a resuscitation assembly according to the invention with the expiration indicator attached to a valve assembly;

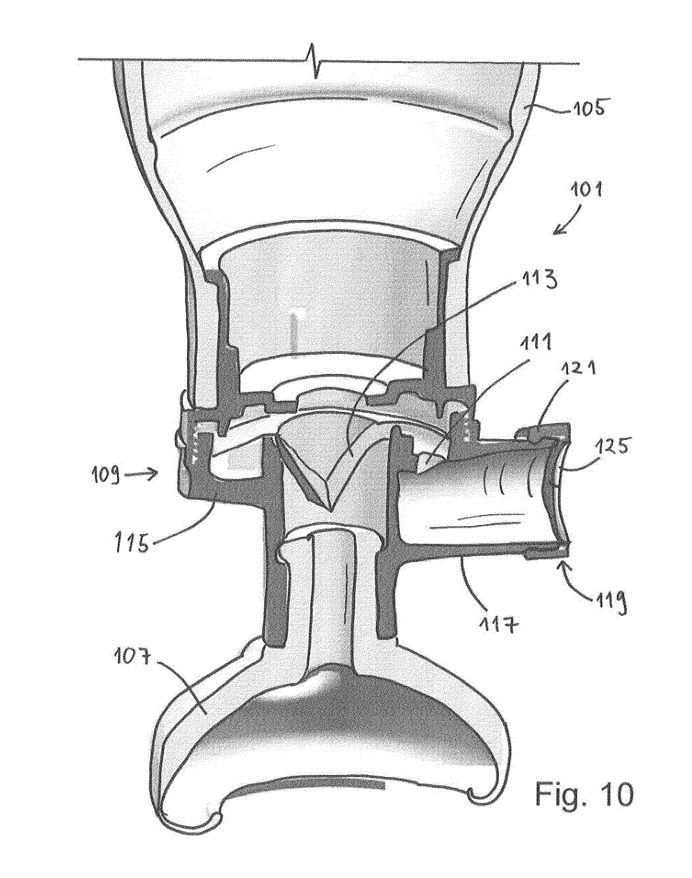

FIG. 10 is a cross section view through the resuscitation assembly of FIG. 9;

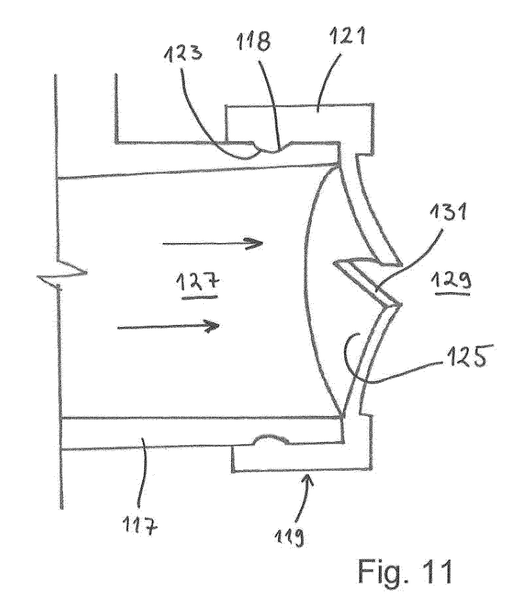

FIG. 11 is an enlarged perspective cross section view of the expiration indicator in an open state;

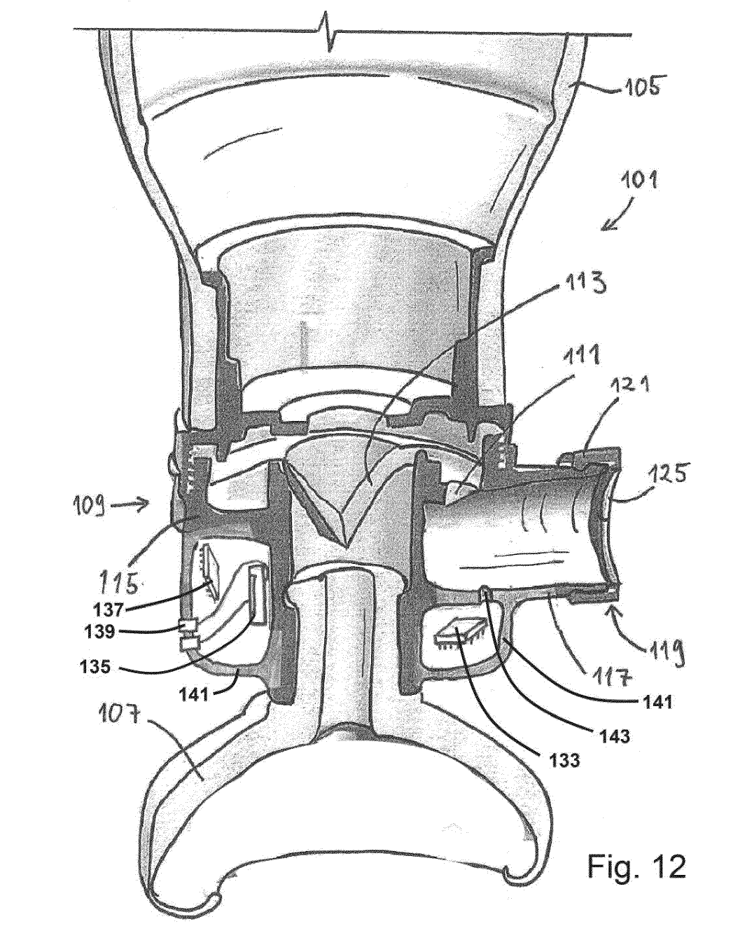

FIG. 12 is a cross section view corresponding to FIG. 10, however showing another embodiment;

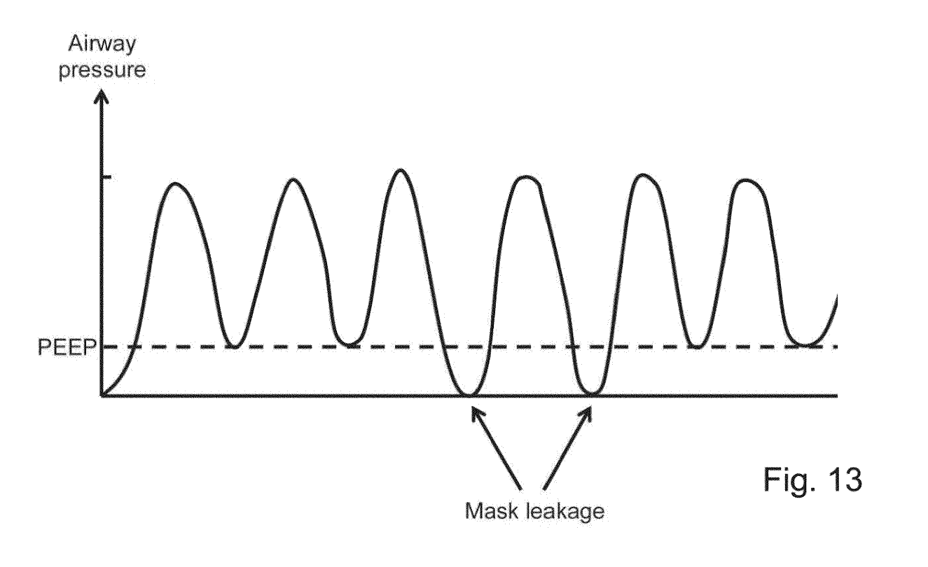

FIG. 13 is a diagram showing measured pressures on the patient side of a PEEP valve;

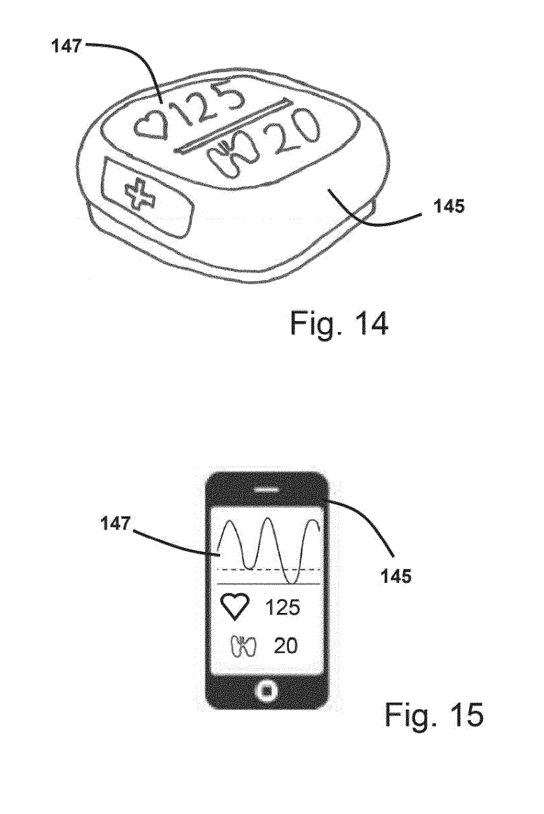

FIG. 14 is a principle view of a remote receiver with a display showing measured pressure values; and

FIG. 15 is a principle view of a remote receiver which in this embodiment is a smart phone.

FIG. 1 is a side view of a resuscitation assembly known from the prior art being used on a patient 3. The resuscitation assembly 1 has a flexible bag 5, a mask 7, and a valve assembly 9 arranged between the flexible bag and the mask.

FIG. 2 is a cross section view of parts of a prior art type resuscitation assembly 1 corresponding to the one in FIG. 1. The valve assembly 9 comprises an exhalation valve 11 and an inflation valve 13. The exhalation valve 11 will open only when air flows out of from the patient 3 and will close when air is inflated into the patient 1. The inflation valve 13, on the other hand, will open only when air is inflated from the flexible bag 5 and into the mask 7, and hopefully into the lungs of the patient, and will close when air flows out from the patient 3. These functions are known to the person skilled in the art and will not be explained in further detail.

FIG. 3 and FIG. 4 show a valve assembly 109 provided with an expiration indicator 119, being part of a resuscitation assembly 101 of an embodiment according to the present invention. The valve assembly 109 corresponds in many respects to the valve assembly 9 shown in FIG. 2. However, in addition to an exhalation valve 111 and inflation valve 113 (see also FIG. 10), the valve assembly 109 shown in FIG. 3 and FIG. 4 also exhibits said expiration indicator 119.

The valve assembly 109 exhibits a valve assembly housing 115 out from which a pipe stub 117 extends. In this embodiment, the expiration indicator 119 is in form of a cap that can be fitted onto the pipe stub 117. In FIG. 4 the expiration indicator 119 is shown arranged on the pipe stub 117.

The inner bore of the pipe stub 117 is in fluid connection with the exhalation valve 111 in the valve assembly 109. Thus, air being exhaled from the patient is guided into the pipe stub 117 and to the expiration indicator 119.

FIG. 5 and FIG. 6 show enlarged perspective views of the expiration indicator 119.

FIG. 7 and FIG. 8 show cross section views of the expiration indicator 119. The expiration indicator 119 exhibits a circular body 121 with an inner diameter that fits onto the pipe stub 117. In the inner diameter of the circular body 121 there is arranged a circular protrusion 123 that is adapted to engage with a facing circular groove 118 on the pipe stub 117. This is to ensure that the expiration indicator 119 remains on the pipe stub 117 during use. The expiration indicator 119 can thus be releasably connected to the valve assembly 109.

The expiration indicator 119 has one patient side 127 that faces the air coming from the lungs of the patient 3, and one ambience side 129 that faces the ambient air or the surroundings.

The expiration indicator 119 further comprises a flexible sheet part 125 which is shaped like a dome or a curved or convex sheet. The convex shaped face of the sheet part 125 faces towards the patient side 127, whereas the opposite concave shaped face faces towards the ambience side 129. When the pressure on the patient side 127 reaches a predetermined value with respect to the pressure on the ambience side 129, typically a pressure of 4 to 8 cm H₂O, the pressure will force the sheet part 125 to deflect. As will be appreciated by the skilled person, this pressure can be predetermined by choice of thickness, material, diameter, and shape (curvature) of the flexible sheet part. The said pressure can of course also be less or larger than 4 to 8 cm H₂O.

A through slit 131 is arranged in the flexible sheet part 125. When the expiration indicator 119 is in a closed state, as shown in FIG. 7, the facing walls of the slit 131 remains in contact and do not let air pass through the slit 131. However, when the predetermined pressure exists on the patient side 127 and the flexible sheet part 125 deflects, the facing walls of the slit 131 will move away from each other and thus open for flow of air through the slit 131. This open state is shown in FIG. 8.

When the air flow and pressure diminishes, the flexible sheet part 125 will return to the closed state. That is, it will return to the closed state when the pressure on the patient side 127 returns to below approximately the said predetermined pressure. In this way, there will remain a pressure within the lungs of the patient during and after exhalation. This is one of two main operational functions to be fulfilled with the expiration indicator 119.

Furthermore, the user of the resuscitation assembly 101 according to the present invention will be able to see whether or not the expiration indicator 119 is in the open or closed state. That is, the different state of the open or closed slit 131 is clearly visible. If he can see that it opens for every ventilation cycle, he will know that air has been ventilated into the lungs of the patient, since only exhaled air will flow through the exhalation valve 111 and thus through the expiration indicator 119. This function fulfills the second main operational function of the expiration indicator 119 when in use on a patient 3.

FIG. 9 shows the entire resuscitation assembly 101 according to an embodiment of the present invention, with the expiration indicator 119 attached to the valve assembly 109.

FIG. 10 is a cross section perspective view through parts of the resuscitation assembly 101. In this drawing the expiration indicator 119 is in the closed state.

FIG. 11 is en enlarged cross section perspective view of the pipe stub 117 and the expiration indicator 119 in the open state. In contrast to the shape of the flexible sheet part 125 in FIG. 10, in FIG. 11 the flexible sheet part 125 bulges towards the ambience side 129 and the slit 131 is opened.

FIG. 12 corresponds in many respects to FIG. 10 described above. In the embodiment shown in FIG. 12 however, a pressure sensor 133 is arranged within a measuring housing 141 attached to the valve assembly housing 115. The pressure sensor 133 is in fluid communication with the patient side 127 of the expiration indicator 119, through an aperture 143 in the pipe stub 117.

The measuring housing 141 may advantageously extend entirely about a portion of the valve assembly housing 115, but may also extend only partially about the valve assembly housing 115.

Within the measuring housing 141 there is also arranged a signal transmission device 137 which is functionally connected to the pressure sensor 133. The signal transmission device 137 is adapted to transmit measured pressure values, as measured by the pressure sensor 133, wirelessly to a remote receiver (cf. FIG. 14 and FIG. 15). Preferably within the measuring housing 141 there may also be arranged electrical circuitry suitable for operating the pressure sensor 133 and the signal transmission device 137, including a signal conditioning unit and battery charging unit.

A battery 135 is adapted to provide electric power and is adapted to be charged through a pair of electrodes 139 arranged in a wall part of the measuring housing 141. In FIG. 12 a pair of conductors is indicated, running from the electrodes 139 to the battery 135. However, such conductors could also be extended from the electrodes to the said battery charging unit, according to what the skilled person would find appropriate for the specific embodiment.

The pressure sensor 133, battery 135, signal transmission device 137, and electrodes 139 are only schematically illustrated for the sake of explaining this embodiment of the invention.

FIG. 13 illustrates a diagram of measured pressure values as a function of time.

At a lower portion of the pressure scale, the positive end expiratory pressure (PEEP) value for the expiration indicator 119 is indicated with a dashed line. By having the measured pressure values, as measured by the pressure sensor 133, displayed to the person performing the ventilation, such as on a computer display or other type of display, he or she may monitor the actual pressure. If a leakage occurs between the mask 107 and the face of the patient, this can be detected as the pressure diagram shows that actual pressure is below the desired positive end expiratory pressure. In FIG. 13 such an occurrence of mask leakage is shown in two succeeding cycles. The person performing the ventilation has detected the leakage and re-established a sealing contact between the mask 107 and the face, resulting in a good next ventilation cycle.

FIG. 14 and FIG. 15 schematically illustrate two different types of remote receivers 145. FIG. 14 shows a dedicated remote receiver 145 comprising a signal receiving unit (not shown) and a display. In addition to receiving signals with pressure value information, the remote receiver 145 is also adapted to receive signals carrying information of heart rate. Thus, the display is adapted to display both a hart rate as well as the positive end expiratory pressure.

The remote receiver 145 shown in FIG. 15 is a smart phone adapted to wirelessly receive and to display the pressure values. It also displays heart rate. In addition its display is adapted to show a pressure value diagram, such as the one shown in FIG. 13, making the person performing ventilation able to easily detect a malfunctioning sealing between the mask and the face of the patient.

The remote receiver may also be of another type than what is shown herein, for instance a tablet or a computer. It should also be noted that the remote receiver, receiving the signals from the signal transmission device 137, may comprise more components. For instance, a separate remote receiver may be connected to a computer, wherein the first receives the signals and the latter displays the pressure values, possibly also other values.

The remote receiver 145 has interpretation software which can give objective feedback about ventilation performance. Such feedback may include mask leak, desired ventilation rate, applied pressure and PEEP. The remote receiver 145 may also receive data from other devices, for instance it can receive heart rate data from a heart rate sensor, as discussed above, since heart rate is an essential parameter for newborn resuscitation. The receiver may also receive data from a chest compression sensor, since chest compressions and ventilation together are essential parameters when resuscitating children and adults. In addition, recorded data may be used in quality improvement programs.

By getting objective feedback, the person performing ventilation can adapt to the desired pressure and ventilation rate, ensure that PEEP is present and adjust the mask to improve mask seal if necessary. This will improve the quality and effectiveness of the face mask ventilation.

1. A resuscitation assembly comprising:
a patient mask;
a ventilation bag;
an inflation valve;
an exhalation valve; and
an expiration indicator; and
wherein the expiration indicator is a positive end expiratory pressure valve and the expiration indicator is a slit valve that comprises a slit in a flexible sheet part.

2. The resuscitation assembly according to claim 1, wherein:
the expiration indicator has a patient side and an ambience side; and that
the flexible sheet part exhibits a convex face that faces the patient side; and
the flexible sheet part is adapted to deflect towards the ambience side at a pressure on the patient side in such way that a visible gap is formed in the slit.

3. The resuscitation assembly according to claim 1, comprising:
a valve assembly arranged between the patient mask and the ventilation bag; and
wherein the expiration indicator is arranged to the valve assembly.

4. The resuscitation assembly according to claim 3, wherein the valve assembly exhibits a pipe stub adapted to receive the expiration indicator.

5. The resuscitation assembly according to claim 1, wherein the expiration indicator is in the form of a cap which is adapted to be releasably arranged to a receiving connecting piece of the resuscitation assembly.

6. The resuscitation assembly according to claim 5, wherein said cap exhibits a circular body with at least one of a circular protrusion and a circular groove arranged in the inwardly facing face of said circular body.

7. The resuscitation assembly according to claim 1, comprising a pressure sensor adapted to measure pressure on a patient side of the expiration indicator.

8. The resuscitation assembly according to claim 7, characterized in that wherein measured pressure values, as measured by said pressure sensor, is adapted to be communicated wirelessly to a remote receiver, as the resuscitation assembly comprises a signal transmission device and a battery.

9. The resuscitation assembly according to claim 8, wherein the remote receiver is adapted to receive and to display a plurality of measured parameters, such as said pressure values, heart rate values, and chest rise values.

10. A resuscitation assembly comprising:
a patient mask;
a ventilation bag;
an inflation valve;
an exhalation valve; and
a positive end expiratory pressure valve; and
a pressure sensor adapted to measure pressure on the patient side of the positive end expiratory pressure valve.

11. The resuscitation assembly according to claim 10, comprising a signal transmission device adapted to transmit measured pressure values to a remote receiver.

12. The resuscitation assembly according to claim 10, comprising further comprises a display adapted to display current pressure values, as measured by said pressure sensor.
